(19) United States
(12) Patent Application Publication
(10) Pub. No.: US 2015/0088109 A1
(43) Pub. Date: Mar. 26, 2015

(54) APPARATUS AND METHOD FOR PAIN REDUCTION DURING A TREATMENT APPLICATION

(71) Applicant: LUMENIS LTD., Yokneam Ilit (IL)

(72) Inventor: Shiri Salomon, Ramat- Hasharon (IL)

(21) Appl. No.: 14/492,987

(22) Filed: Sep. 22, 2014

Related U.S. Application Data
(60) Provisional application No. 61/881,334, filed on Sep. 23, 2013.

(51) Int. Cl. A61N 1/36; A61B 18/18; A61B 18/20; A61N 7/00; A61B 18/14

(52) U.S. Cl. CPC A61N 1/36021; A61N 7/00; A61B 18/14; A61B 18/203; A61B 18/18; A61B 2018/1807
USPC 606/13; 607/3; 606/169; 606/41

(57) ABSTRACT

A treatment apparatus comprising a handpiece, the handpiece having a primary treatment element and a contact surface, at least one electrode located at the contact surface, and a connection element to connect the at least one electrode to a controller to provide analgesia.

### FIELD OF THE INVENTION

The present application relates to an apparatus and method for reducing pain and discomfort during a treatment application comprising a handpiece, particularly but not exclusively for cosmetic treatment, and a method of operating a treatment apparatus.

### BACKGROUND TO THE INVENTION

Aesthetic or cosmetic procedures are often based on the delivery of electromagnetic energy to the skin or tissue to be treated. Examples of such procedures include: fat reduction, skin tightening, dermal microabrasion, treatment of pigmentation disorders or removal of tattoos, and hair removal, amongst many others. Delivery of the electromagnetic energy to the right volume of skin or tissue can be performed using laser light, radio frequency (‘RF’) signals, and ultrasound.

A disadvantage of such treatments or procedures is that they may be accompanied with patient discomfort, if not pain as well. Patients who suffer from discomfort may often result in low adherence to the treatment regime over a period of time and may be reluctant to complete the session or the entire treatment protocol. It is known to provide analgesia before, during or after the treatment, for example using a topical cream, applying a cold-transmitting device to the skin or even administering oral painkillers, but these are not always satisfactory.

### SUMMARY OF THE INVENTION

According to a first aspect of the invention, a treatment apparatus has a handpiece having a primary treatment element and a contact surface for contacting a patient tissue surface, at least one electrode located on the contact surface, and a controller connected to the at least one electrode. The controller causes the treatment apparatus to provide: (a) a treatment signal to the primary treatment element to provide treatment to the patient tissue surface and (b) an electric current to the at least one electrode to provide analgesia to the patient tissue surface.

According to another aspect, the primary treatment element includes at least one of an ultrasonic transducer, a radio-frequency source, a light source and a laser element.

According to another aspect, the treatment apparatus may include one of: two electrodes, four electrodes or six electrodes or more pairs of electrodes.

According to another aspect, the treatment apparatus the treatment signal and the electric current are provided substantially simultaneously.

In another aspect, the treatment signal is provided either prior to or after the electric current.

In another aspect, the treatment apparatus the electric current is provided from about 2 to about 5 seconds before the treatment signal.

In an aspect, the treatment apparatus the electric current is provided during the time that the treatment signal is provided and following the end of the treatment signal.

In an aspect, the treatment apparatus the electric current has an amplitude in the range 1 to 50 mA into a 1 kΩ load.

In yet another aspect, the electric current is pulsed, each pulse having a duration in the range 10 to 1000 μs.

In another aspect, each pulse has a duration in the range 100 to 200 μs.

In another aspect, the pulses have a frequency in the range 1 to 250 Hz.

In another aspect, the pulses having a frequency in the range of 50 to 80 Hz.

In another aspect, the pulses have a repetition rate of 1 Hz.

In another aspect, the treatment apparatus has at least one electrode located at or near the treatment area on the patient tissue surface.

In another aspect, the electric current provided causes selective activation of AB fibers in the patient nervous system, thereby providing analgesia by way of transcutaneous electrical nerve stimulation (TENS) stimulation.

In another aspect, the primary treatment element is a source of one of: laser energy or intense pulsed light (IPL) energy, the energy being delivered through an application tip placed onto or spaced from the patient skin surface and the electric current being delivered to two electrodes placed in contact with the patient tissue surface, the electrodes being located one on each side of the application tip.

In another aspect, the treatment apparatus the primary treatment element is a source of one or more of ultrasonic transducers or RF electrodes or both, the energy from the one or more of ultrasonic transducers or RF electrodes being delivered within a cavity to the patient tissue surface positioned with the cavity, the electric current being delivered to two or more electrodes positioned within the cavity and in contact with the patient tissue surface within the cavity.

According to a second aspect of the invention, a method of operating a treatment apparatus includes: providing a handpiece, the handpiece having a primary treatment element and a contact surface for contacting a patient tissue surface and at least one electrode located on the contact surface. The treatment apparatus further provides a controller connected to the at least one electrode. The controller is structured to cause the treatment apparatus to provide: (a) a treatment signal to the primary treatment element to provide treatment to the patient tissue surface and (b) an electric current to the at least one electrode to provide analgesia to the patient tissue surface including the step of placing the contact surface adjacent a skin surface; and, supplying a signal from the controller to provide the treatment signal and the electric current to the patient tissue surface.

In an aspect, the primary treatment element is at least one of an ultrasonic transducer, a radio-frequency source, a light source and a laser element.

In another aspect, the electric current provided causes selective activation of AB fibers in the patient nervous system, thereby providing analgesia by way of transcutaneous electrical nerve stimulation (TENS) stimulation.

### BRIEF DESCRIPTION OF THE DRAWINGS

Embodiments of the invention may be described by way of example only with reference to the accompanying drawings, wherein;

FIG. 1 is a diagrammatic illustration of a known treatment apparatus,

FIG. 2 is a diagrammatic illustration of a treatment apparatus embodying the present invention,

FIG. 3 is a diagrammatic illustration of a signal generated by a controller of the apparatus of FIG. 2,

FIG. 4a is a diagrammatic illustration of part of a further treatment apparatus embodying the present invention,

FIG. 4b is an end view from below of the part of FIG. 4a,

FIG. 5 is a diagrammatic illustration of the operation of the treatment apparatus of FIG. 4a, and

FIG. 6 is a perspective view of a further treatment apparatus embodying the present invention.

### DETAILED DESCRIPTION OF THE PREFERRED EMBODIMENTS

With specific reference now to the drawings in detail, it is stressed that the particulars shown are by way of example and for purposes of illustrative discussion of the preferred embodiments of the present invention only, and are presented to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated n the drawings. The invention is applicable to other embodiments or of being practiced or carried out in various ways. Also, it is to be understood that the phraseology and terminology employed herein is for the purpose of description and should not be regarded as limiting.

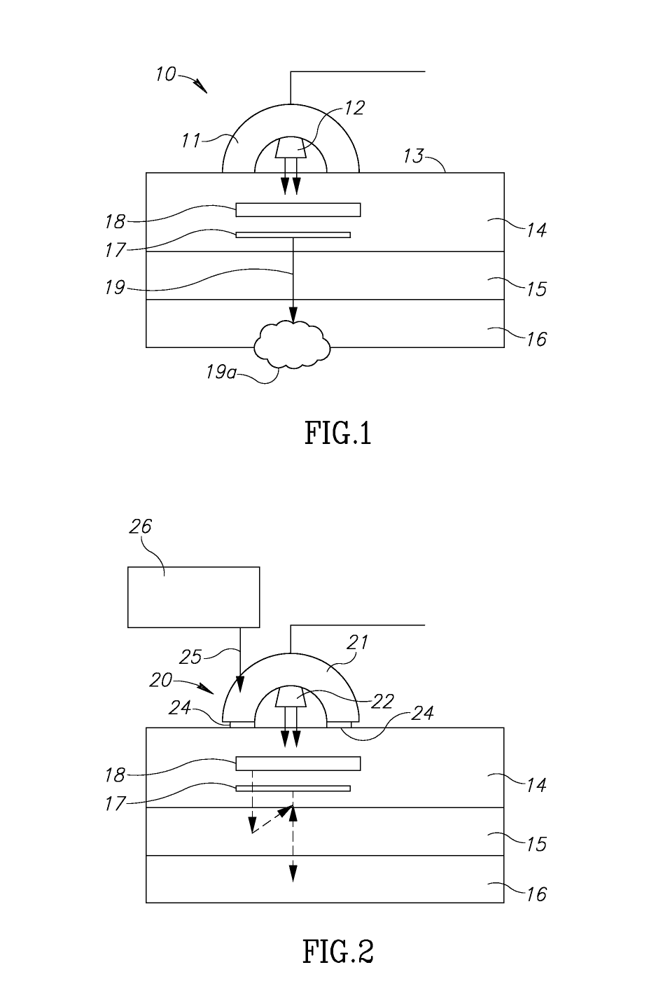

With reference to FIG. 1, a treatment apparatus of known type for aesthetic or cosmetic treatment is generally shown at 10. The treatment apparatus 10 has a handpiece 11 having a treatment element 12. The treatment element 12 may be any known energy source for treatment, such as at least one of an ultrasonic transducer, light source, laser light source or radiofrequency source. In use, the handpiece is placed against a skin surface 13 and the treatment element 12 controlled to perform the desired procedure.

The nervous system of a patient is shown diagrammatically, with the skin layers shown at 14, the spinal cord connection illustrated at 15 and the brain at 16. The nervous system includes small diameter Aδ and C fibers generally illustrated at 17 and Aβ fibers shown at 18 (large diameter fibers which may be activated by touch). When the nociceptive Aδ and C fibers are stimulated, in particular by a stimulus which may be a result of damage, the stimulus is conveyed through the spinal cord as shown by arrow 19, resulting in a sensation of pain as illustrated at 19a.

Accordingly, when the treatment element 12 is actuated, energy is supplied to the skin or tissue 14, causing localized (and desirable) damage, the Aδ and C fibers are stimulated and a sensation of pain results.

Referring now to FIG. 2, a treatment apparatus embodying the present invention is shown at 20, comprising a handpiece 21 having a treatment element 22. The handpiece 21 has a contact surface 23 for placing against or in proximity to a skin surface 13 of a patient undergoing treatment. At least one electrode 24 is disposed on the contact surface 23, such that the electrode 24 will engage the skin surface 13 during treatment. A connection 25 is provided to connect the electrode 24 to a controller 26, such that a signal can be sent to the electrode 24. Preferably at least 2 electrodes 24 are provided such that current passes through the skin 14 in a controlled manner, through or near the area to be treated.

By transmitting a signal to the electrodes 24, analgesia or pain relief may be induced, using transcutaneous electrical stimulation., known in the art by the abbreviation TENS. When transcutaneous electrical stimulation is delivered prior to and/or concomitant to stimulation of Aδ and C fibers 17, selective activation of the Aβ fibers 18 occurs. The activation of the Aβ fibers 18, activates in turn inhibitory spinal interneurons which prevent the passage of information delivered by activated Aδ and C from reaching to the brain which results in pain inhibition or smaller pain sensation. In the known “gate” theory of pain, inhibitory and excitory synapses in the central nervous system regulate the passage of information to the brain. By stimulating the Aβ fibers, inhibitory synapses are activated, which prevent the transmission of further signals to the brain, including those from the activated Aδ and C fibers 17.

To provide analgesia, it is apparent that the electrodes 24 should be located close to the area or volume treated by the treatment element 22. A signal is sent to the electrodes 24 before treatment begins, preferably from about 2 to about 5 seconds beforehand, and continues during (and optionally after) the treatment procedure.

The signal characteristics may be adapted to as appropriate or effective. The waveform may be monophasic (i.e. oscillating between 0V and a positive or negative voltage) or biphasic (i.e. oscillating between positive and negative voltages). If the magnitudes of the positive and negative voltages are equal, the signal is described as symmetrical biphasic, otherwise it is referred to as asymmetric al biphasic.

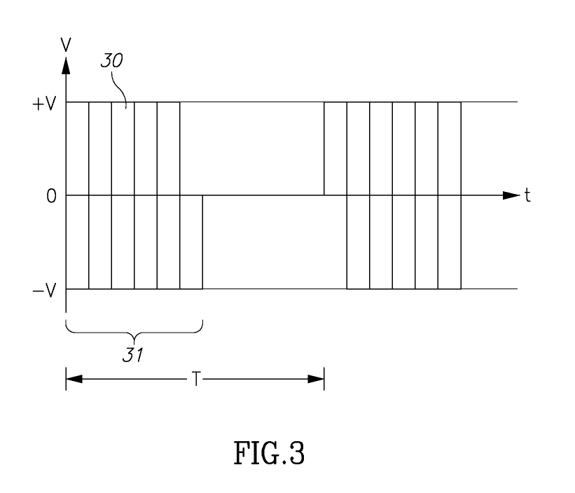

An example waveform is shown in FIG. 3. The signal comprises square pulses 30 oscillating between equal positive and negative voltages (i.e. the signal is symmetrical biphasic). The pulses are grouped into bursts 31, which have a repetition time T. The waveform is purely illustrative: there may be any number of pulses 30 in a burst 31, the pulses may have any appropriate shape and so on. In the present example, the pulse amplitude may be in the range of 1 to 50 mA into a 1 kΩ load. The pulse duration may be in the range 10 to 10000, and preferably in the range 100 to 200 μS as it is believed this provides better selective activation of the Aβ fibers 18. The pulse frequency in is the range 1 to 250 Hz and preferably 50 to 80 Hz. The repetition rate, 1/T, is about 1 Hz.

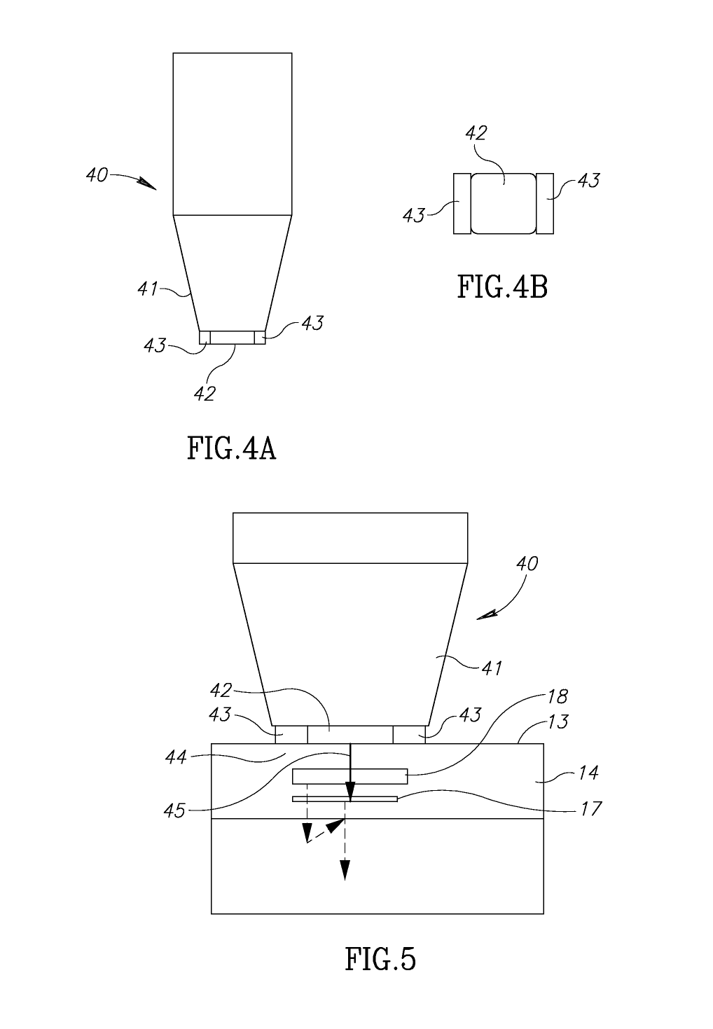

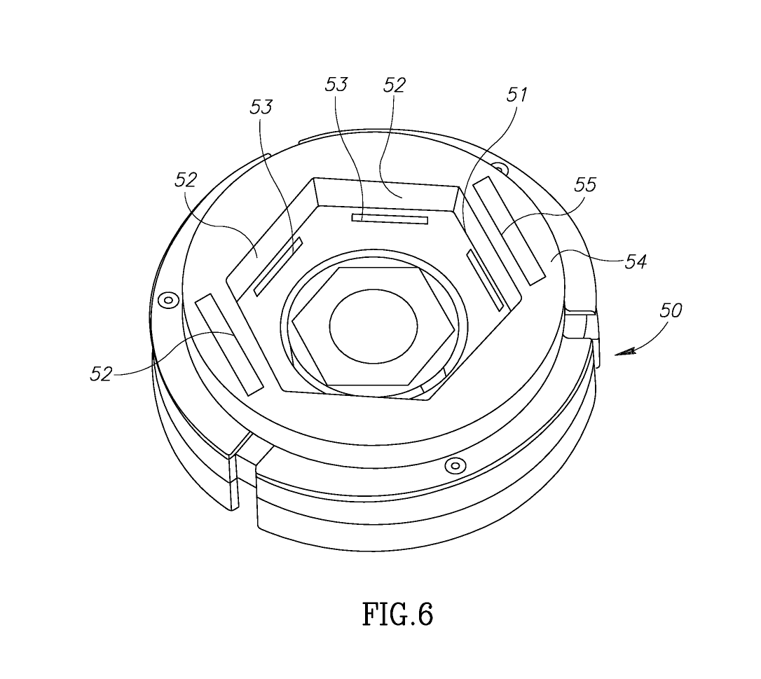

A further example of part of a handpiece of a treatment apparatus is shown at 40 in FIGS. 4a and 4b. The tip 41 of the handpiece comprises a laser source 42, in the present example a sapphire window transmitting laser light with a wavelength of 805 nm. Electrodes 43 are located either side of the window 42. As illustrated in FIG. 5, in operation the tip 41 is placed adjacent the skin surface 13. By supplying a signal to the electrodes 43, a current passes through the skin as illustrated at 44, activating Aβ fibers 18. When laser light is transmitted into the skin as shown at 45, the Aδ and C fibers 17 are stimulated but due to the gate effect caused by stimulation of the Aβ fibers 18, any resulting pain sensation is reduced or inhibited.

A further example of a handpiece is shown at 50 in FIG. 6. In this example, the handpiece 50 comprises a cavity 51 into which tissue is drawn for treatment. The handpiece includes a plurality of treatment elements, including ultrasonic transducers 52 and RF electrodes 53 arranged around the cavity 51. The handpiece 50 has a contact surface 54 for positioning against the patient's skin. Electrodes 55 are positioned on the contact surface, and a signal may be transmitted from a controller to the electrodes 55 to provide pain relief as discussed above.

It will be apparent that the treatment element may be one or more of an ultrasonic transducer, a radio-frequency source, a light source and a laser element, and a handpiece may have one or more types of treatment elements. Any appropriate number of electrodes may be provided depending on the area or volume of tissue where treatment occurs, for example 2 as shown herein, 4, 6, or more as needed.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

Meanings of technical and scientific terms used herein are to be commonly understood as by one of ordinary skill in the art to which the invention belong, unless otherwise defined.

What we claim is:

1. A treatment apparatus comprising;
a handpiece, the handpiece having a primary treatment element and a contact surface for contacting a patient tissue surface;
at least one electrode located on the contact surface; and,
a controller connected to the at least one electrode, the controller causing the treatment apparatus to provide: (a) a treatment signal to the primary treatment element to provide treatment to the patient tissue surface at the contact surface, and (b) an electric current to the at least one electrode to provide analgesia to the patient tissue surface.

2. The treatment apparatus according to claim 1 wherein the primary treatment element comprises at least one of an ultrasonic transducer, a radio-frequency source, a light source and a laser element.

3. The treatment apparatus according to claim 1 comprising one of: two electrodes, four electrodes, six electrodes or more pairs of electrodes.

4. The treatment apparatus of claim 1 wherein the treatment signal and the electric current are provided substantially simultaneously.

5. The treatment apparatus of claim 1 wherein the treatment signal is provided either prior to or after the electric current.

6. The treatment apparatus of claim 1 wherein the electric current is provided from about 2 to about 5 seconds before the treatment signal.

7. The treatment apparatus of claim 6 wherein the electric current is provided during the time that the treatment signal is provided and following the end of the treatment signal.

8. The treatment apparatus according to claim 1 wherein the electric current has an amplitude in the range 1 to 50 mA into a 1 kΩ load.

9. The treatment apparatus according to claim 1 wherein the electric current is pulsed, each pulse having a duration in the range 10 to 1000 μs.

10. The treatment apparatus according to claim 9 wherein each pulse has a duration in the range 100 to 200 μs.

11. The treatment apparatus according to claim 9 wherein the pulses have a frequency in the range 1 to 250 Hz.

12. The treatment apparatus according to claim 9 wherein the pulses have a frequency in the range of 50 to 80 Hz.

13. The treatment apparatus according to claim 9 wherein the pulses have a repetition rate of 1 Hz.

14. The treatment apparatus of claim 1 wherein the at least one electrode is located at or near the treatment area on the patient tissue surface.

15. The treatment apparatus of claim 1 wherein the electric current provided causes selective activation of AB fibers in the patient nervous system, thereby providing analgesia by way of transcutaneous electrical nerve stimulation (TENS) stimulation.

16. The treatment apparatus of claim 1 wherein the primary treatment element is a source of one of laser energy or intense pulsed light (IPL) energy, the energy being delivered through an application tip placed onto or spaced from the patient skin surface and the electric current being delivered to two electrodes placed in contact with the patient tissue surface, the electrodes being located one on each side of the application tip.

17. The treatment apparatus of claim 1 wherein the primary treatment element is a source of one or more of ultrasonic transducers or RF electrodes or both, the energy from the one or more of ultrasonic transducers or RF electrodes being delivered within a cavity to the patient tissue surface positioned with the cavity, the electric current being delivered to two or more electrodes positioned within the cavity and in contact with the patient tissue surface within the cavity.

18. A method of operating a treatment apparatus comprising:
providing a handpiece, the handpiece having a primary treatment element and a contact surface for contacting a patient tissue surface and at least one electrode located on the contact surface;
providing a controller connected to the at least one electrode, the controller structured to cause the treatment apparatus to provide: (a) a treatment signal to the primary treatment element to provide treatment to the patient tissue surface and (b) an electric current to the at least one electrode to provide analgesia to the patient tissue surface; and,
wherein the method comprises the step of placing the contact surface adjacent a skin surface; and,
supplying a signal from the controller to provide the treatment signal and the electric current to the patient tissue surface.

19. The method of claim 18, wherein the primary treatment element comprises at least one of an ultrasonic transducer, a radio-frequency source, a light source and a laser element.

20. The method of claim 18, wherein the electric current provided causes selective activation of AB fibers in the patient nervous system, thereby providing analgesia by way of transcutaneous electrical nerve stimulation (TENS) stimulation.
